(19) United States
(12) Patent Application Publication
(10) Pub. No.: US 2010/0092537 A1
(43) Pub. Date: Apr. 15, 2010

(54) SURGICAL IMPLANT COMPOSITE MATERIALS AND KITS AND METHODS OF MANUFACTURE

(76) Inventor: Maria Strömme, Uppsala (SE)

Correspondence Address: DRINKER BIDDLE & REATH (DC), 1500 K STREET, N.W., SUITE 1100, WASHINGTON, DC, 20005-1209, US

(21) Appl. No.: 12/514,272

(22) PCT Filed: Nov. 6, 2007
(86) PCT No.: PCT/IB2007/054505

§ 371 Date: Nov. 19, 2009

Related U.S. Application Data
(60) Provisional application No. 60/857,892, filed on Nov. 10, 2006.

(51) Int. Cl. A61F 2/00; B05D 7/00

(52) U.S. Cl. 424/423; 427/2.24

(57) ABSTRACT

A surgical implant composite material comprises a surgical implant substrate and a thin film coating deposited on the substrate, the thin film coating comprising TiO₂₋ₓM_{y}, wherein M is one or more elements which does not adversely effect adherence of the coating to the substrate, y is the sum of the mols of all M elements, 0≦x<2 and 0≦y≦1, and wherein an outermost portion of the thin film coating is crystalline. Kits include a surgical implant composite material and at least one solution of a releasable agent operable to load the releasable agent onto the surgical implant composite material. Methods of forming composite materials comprise depositing a thin film on a substrate.

### FIELD OF THE INVENTION

The present invention is directed to composite materials, for example, surgical implant composite materials, kits including such composite materials, and methods of manufacturing such composite materials.

### BACKGROUND OF THE INVENTION

Metallic implants such as titanium, titanium alloys, stainless steel and Co—Cr alloys are widely used as surgical implants owing to their mechanical strength, biocompatibility, chemical inertness and machinability. Examples of metallic implants include dental implants, cochlear implants, pedicle screws, spinal implants, hip implants, plates, nails, arm and leg amputation implants, and the like. Non-resorbable ceramic implants are also used owing to their mechanical strength, biocompatibility, chemical inertness, and superior aesthetics and high wear resistance. Examples of non-resorbable ceramic implant materials include zirconium dioxide and aluminum oxide. For an overview description of implant materials and their use, reference is made to Ratner et al., Biomaterials Science; An Introduction to Materials in Medicine, 2nd Edition, San Diego (2004).

One of the requirements of implants, wherever they are used in the body, is the ability to form a mechanically stable unit with the surrounding hard or soft tissue. An unstable unit may function less efficiently or cease functioning completely, and/or may induce an excessive tissue response. This may cause patient discomfort and, in certain situations, an unstable implant may need to be surgically removed, for example, owing to little or no tissue in growth to the implant, infection, and/or weak bone formation due to osteoporosis or other diseases.

Several methods have been proposed in the literature to overcome instability owing to insufficient host tissue in growth. The roughness, morphology and/or chemical composition of the implant surface can be modified to promote tissue in growth. For an overview of the state-of-the-art of surface modification of surgical implants reference is made to Brunette et al, Titanium in Medicine, Heidelberg (2001) and Ellingsen et al, Bio-Implant Interface: Improving Biomaterials and Tissue Reactions, CRC Press (2003). The surface roughness of implants is typically controlled via acid etching and/or grit blasting of the surface, see U.S. Pat. No. 6,491,723 and Brunette et al. A roughness of 1 to 5 micrometers has been proven to promote bone in growth. Also heating of the surface using a focused laser has been proposed to create an appropriate surface roughness at specific locations.

A combination of improved surface roughness, morphology and modified composition of titanium implants has been obtained via anodic oxidation of an implant, see WO 2005/055860 and U.S. Pat. No. 6,183,255. The anodic oxidation results in a several micrometer thick porous titanium oxide surface layer, see Lin et al, “Corrosion test, cell behaviour test, and in vivo study of gradient TiO₂ layers produced by compound electrochemical oxidation,” Journal of Biomedical Materials Research, Part A, 78(3):515-22 (2006 Sep. 1). The oxide has been proposed to be used as a carrier of bone morphogenic proteins and peptides to promote bone formation on the implant; see also SE 523,012. It has also been shown that TiO₂₋ₓ films (with x between 0 and 0.35), possibly containing implanted H, Ta or Nb, are blood compatible, and that this type of material can be produced by a plasma immersion ion implantation process. See U.S. Patent Publication No. 2003/0175444 A1, which describes a gradient structure from TiN at the implant interface to TiO₂₋ₓ with a rutile structure at the surface. The TiN layer is added to increase the mechanical properties of the coating. The addition of H, Ta or Nb to the coating is also proposed to be performed using a sputtering method.

Methods to promote bone formation via tailoring the chemical composition of the implant often involve coating of the metal or ceramic surgical implant with a ceramic layer consisting essentially of hydroxylapatite (HA) or calcium phosphate; see Yang et al, “A review on calcium phosphate coatings produced using a sputtering process—an alternative to plasma spraying,” Biomaterials, 26:327-337 (2005). HA is a commonly used bioactive ceramic material and its use is predicated upon its excellent affinity to bone. This property is due to the fact that HA is a major component of the bones and teeth of living animals. Bioactivity, in this context, is defined as interfacial bonding of an implant to tissue by means of formation of a biologically active hydroxylapatite layer on the implant surface, Ducheyne et al, “Bioceramics: material characteristics versus in vivo behaviour,” Annals of the New York Academy of Science, 523 (Jun. 10, 1988). Bioactive materials have been proven to promote bone formation and to form a stable bond to bone, Hench, “Biomaterials: a forecast for the future,” Biomaterials, 19:1419-14239 (1998). A bioactive material may spontaneously form a layer of HA on a surface in vivo when it comes in contact with body fluids. Synthetically produced HA also has bioactive properties and a new layer of carbon rich HA forms on its surface when implanted. Prediction of a material's bioactivity can be made both in vivo and in vitro, Kokubo et al, “How useful is SBF in predicting in vivo bone bioactivity,” Biomaterials, 27:2907-2915 (2006).

In addition to HA and calcium phosphate, a number of other material systems have been shown to be bioactive, e.g. Wollastonite (calcium silicate), see De Aza et al, “Bioactivity of pseudowollastonite in human saliva,” Journal of Dentistry, 27:107-113 (1999); Bioglass, see Hench et al; and Rutile (titanium dioxide) and Anatase (titanium dioxide), see Kim, “Ceramic bioactivity and related biomimetic strategy,” Current Opinion in Solid State & Materials Science, 7:289-299 (2003). Other methods to obtain bioactivity of titanium implants are via chemical treatment in Na—F, see U.S. Pat. No. 5,571,188, and NaOH, see Ma et al, “Biomimetic processing of nanocrystallite bioactive apatite coating on titanium,” Nanotechnology, 14:619-623 (2003).

In summary, techniques that have been proposed to obtain a surface composition that promotes bone in growth include:

1. Coating of the surface with HA or calcium phosphate or other bioactive materials. However, difficulties in the coating process result in low coating adhesion or coating stability, see U.S. Pat. No. 5,480,438, often due to technical difficulties in depositing thin films of complex oxide systems.

2. Chemical treatment of Ti with NaOH or Na—F. These surfaces can then be implanted directly or after soaking in simulated body fluid or phosphate buffered saline to form an “HA layer on the surface; however, the chemical treatment is only suitable for Ti metals.

3. Heat treatment of Ti to form crystalline anatase or rutile, which may then be implanted. However, to achieve crystalline titanium oxide, the treatment temperature needs to be in excess of 400° C., resulting in reduced mechanical strength of the implant. Additionally, this treatment is only suitable for Ti metals.

4. Anodic oxidation of Ti to form anatase or rutile or a mixture thereof.

It has also been proposed that a drug can be encapsulated into a resorbable polymer which is then coated on a metallic implant to allow for drug delivery from the implant surface, see WO 2006/107336. The release kinetics are controlled via the polymer composition, thickness and drug loading. Methods to immobilize bio molecules on implants via amide cross-linking has also been described, see U.S. Patent Publication No. 2006/0193968.

These various methods have limitations in view of versatility and/or durability, and/or in other aspects. Accordingly, there is a continuing need for implant surface modifications for improving implant use, irrespective of the substrate type or roughness.

### SUMMARY OF THE INVENTION

Accordingly, the present invention is directed to composite materials, for example surgical implant composite materials, which overcome various disadvantages of the prior art, and is directed to kits including such composite materials and methods of manufacturing such composite materials.

In one embodiment, the invention is directed to a surgical implant composite material comprising a surgical implant substrate and a thin film coating deposited on the substrate, the thin film coating comprising TiO₂₋ₓM_{y}, wherein M is one or more elements which does not adversely effect adherence of the coating to the substrate, y is the sum of the mols of all M elements, 0≦x<2 and 0≦y≦1, and wherein an outermost portion of the thin film coating is crystalline.

Throughout the present specification, “crystalline” shall be taken to include all phases or mixtures of phases that produce crystalline peaks in an X-ray diffraction measurement. Crystalline structures thus include, but are not limited to, structures that are monocrystalline, polycrystalline, microcrystalline, nanocrystalline, and combinations thereof, as well as structures consisting of crystalline grains, for example, grains larger than 1 nm, embedded in an amorphous matrix. It should be understood that in thin film coatings wherein the crystalline structure is in the form of crystalline grains embedded in an amorphous matrix, the amorphous matrix portion of the coating is not necessarily of the composition TiO₂₋ₓM_{y} as defined and, for example, x is not limited to an upper value less than 2, but may generally be greater than zero, i.e., x≧0. In one such embodiment, the amorphous portion of the thin film coating is of the composition TiO₂₋ₓM_{y} wherein 0<x<3. As will be apparent therefore, the overall composition of the thin film coating may be of an overall composition TiO₂₋ₓM_{y} wherein x may exceed 2.

In another embodiment, the invention is directed to a kit comprising a surgical implant composite material according to the invention and at least one solution of a releasable agent comprising an active pharmaceutical ingredient, ion or bio molecule, or a combination thereof, the solution being operable to load the releasable agent onto the surgical implant composite material upon contact of the solution with the surgical implant composite material.

In yet another embodiment, the invention is directed to methods of manufacturing composite materials. In one embodiment, a method of forming a surgical implant composite material comprises depositing a thin film coating on a surgical implant substrate, the thin film coating comprising TiO₂₋ₓM_{y}, wherein M is one or more elements which does not adversely effect adherence of the coating to the substrate, y is the sum of the mols of all M elements, 0≦x<2 and 0≦y≦1, and wherein an outermost portion of the thin film coating is crystalline.

In another embodiment, a method of forming a composite material comprises depositing a thin film coating on a substrate, the thin film coating comprising TiO₂₋ₓM_{y}, wherein M is one or more elements which does not adversely effect adherence of the coating to the substrate, y is the sum of the mols of all M elements, 0≦x<2 and 0≦y≦1, wherein the thin film coating has a gradient composition through at least a portion of the thin film coating thickness, and wherein the thin film coating is substantially free of oxygen at the substrate surface.

In yet a further embodiment, a method of forming a composite material comprises cleaning and sputter etching a surface of a metallic substrate to remove native oxide, and depositing a thin film coating on the substrate surface, the thin film coating comprising TiO₂₋ₓM_{y}, wherein M is one or more elements which does not adversely effect adherence of the coating to the substrate, y is the sum of the mols of all M elements, 0≦x<2 and 0≦y≦1, and wherein an outermost portion of the thin film coating is crystalline.

Additional embodiments of the invention, along with advantages thereof, will be apparent in view of the following detailed description.

### BRIEF DESCRIPTION OF THE DRAWINGS

The following detailed description will be more fully understood in view of the drawing in which:

FIG. 1 shows the X-ray diffraction (XRD) pattern of the gradient coating of Sample 1 in Example 5, showing the presence of rutile on the surface, the Ti peaks originating from the Ti-base;

FIG. 2 shows the X-ray photoelectron spectroscopy (XPS) oxygen content spectrum for the gradient coating of Sample 1 in Example 5, from 30 sputtering cycles with 18 s/cycle of sputtering, No. 1 on the horizontal axis corresponds to the oxygen content closest to the surface, wherein the oxygen gradient zone is 50 nm while the TiO₂ layer on top of the gradient is 200 nm; and

FIG. 3 shows a scanning electron microscope (SEM) view of the composite material described in Example 6, showing hydroxylapatite (HA) formed in Example 6 on the graded crystalline titanium oxide coating of Sample 1 of Example 5.

The embodiments set forth in the drawing are illustrative in nature and are not intended to be limiting of the invention defined by the claims. Moreover, individual features of the drawing and the invention will be more fully apparent and understood in view of the detailed description.

### DETAILED DESCRIPTION

The present invention is directed to composite materials, surgical implants, kits, and methods of manufacturing composite materials. The following detailed description shows the best currently contemplated modes of carrying out the invention. The description is not to be taken in a limiting sense, but is made for the purpose of illustrating the general principles of the invention and the best mode for practicing the invention.

The composite materials according to the present invention comprise a substrate and a thin film coating deposited on the substrate. The thin film coating comprises TiO₂₋ₓM_{y}, wherein M is one or more elements which does not adversely effect the adherence of the coating to the substrate, y is the sum of the mols of all of M elements, 0≦x<2 and 0≦y≦1. The thin film coating exhibits high adhesion to the substrate and therefore is advantageous for use in various applications.

In one embodiment of the invention, the substrate comprises a surgical implant. Surgical implants are widely used in, e.g., otology, orthopedics, dentistry treatments of bone or soft tissue defects, and as cardio vascular implants, and the surgical implant substrate for use in the present invention may take any implant form known in the art. The substrate, for example, the surgical implant substrate, may be formed of any suitable material, including, but not limited to, metals, e.g. titanium, titanium alloys, stainless steel, and Co—Cr alloys, ceramics, e.g., zirconium dioxide and aluminum oxide, and polymers. Thus, the composite materials according to the invention are versatile and may be used in various applications, including, but not limited to, surgical implants, owing to the variety of substrate compositions which may be employed therein. In the following description, reference is made to surgical implant substrates. It will be appreciated however that the substrate may be suitable for use in other applications and therefore that the present description is not limited to composite materials employing surgical implant substrates.

The surgical implant substrate is coated by depositing a thin film coating on the substrate surface. The coating comprises titanium and oxygen, and optionally, M, wherein M is one or more elements that do not adversely effect adherence of the coating to the substrate, i.e., that together with Ti provides a material which has good adherence to the implant surface. For example, M may include, but is not limited to, Ag, I, Si, Ca, Zr, Hf, P, C, N, or any combination thereof. In a specific embodiment, M is nitrogen or carbon or a combination of the two. The stoichiometric ratio between Ti and M, can vary within the ranges of TiO₂₋ₓM_{y}, wherein y is the sum of the mols of all M elements, 0≦y≦1. In one embodiment, Ti is the dominating part of the combination.

The thin film coating may be formed to any desired thickness. The thickness of the thin film coating may be selected based on the intended composite material application. In one embodiment, the thin film coating has a thickness less than about 100 μm, or more specifically, less than about 50 μm. In a more specific embodiment, the thin film coating has a thickness less than about 30 μm, or more specifically, less than about 10 μm. In a further embodiment, the thin film coating has a thickness less than about 1 μm, or more specifically, less than about 500 nm.

In one embodiment, the thin film coating has a gradient composition through at least a portion of the thin film coating thickness. In a specific embodiment, the thin film coating is substantially free of oxygen at the substrate surface. Thus, in one further embodiment, the thin film coating is substantially pure Ti metal at the substrate surface. Further, in a specific embodiment, the gradient composition increases in oxygen content in a direction extending from the substrate toward the thin film coating surface. The gradient composition may vary in any manner, for example, monotonically or in a stepwise manner. Further, the gradient composition may vary monotonically in a linear, parabolic, parallel or exponential manner.

In a further embodiment, the thin film coating comprises TiO₂ as the outermost part of the coating, and the thin film coating has a gradient composition through at least a portion of the thin film coating thickness. In a specific embodiment, the coating adjacent the substrate surface is substantially Ti and the coating composition contains an increasing oxygen concentration towards the coating surface, finally reaching a composition consisting substantially of TiO₂ as the outermost part. However, indicated amounts of one or more M elements are acceptable as long as they do not interfere with the substrate adhesion of the coating and are typically included to provide improved substrate adhesion and/or coating functionality such as bioactivity, mechanical stability, surface configuration, i.e., porosity, surface charge, or the like.

When the thin film coating includes a gradient composition, the gradient composition may comprise from 99% to 0.01% of the thin film coating thickness. In a more specific embodiment, the gradient composition may comprise less than about 90% of the thin film coating thickness. In a further embodiment, the gradient composition comprises at least about 10% of the thin film coating thickness. In a specific embodiment, the gradient composition has a thickness of greater than about 7 nm, more specifically greater than about 15 nm, and even more specifically greater than about 40 nm, and/or a thickness less than about 30 μm, more specifically less than about 1 μm, and even more specifically less than about 200 nm. In a further specific embodiment, the gradient composition has a thickness of from about 40 nm to 200 nm.

In one embodiment, the phase composition of the outermost part of the coating is mainly crystalline, for example rutile or anatase or combinations thereof. The thin film coating is provided on the substrate by deposition, and the crystallinity may be provided by controlling the deposition method, i.e., by using chemical vapor deposition (CVD), i.e., laser chemical vapour deposition or low temperature chemical vapour deposition, physical vapour deposition (PVD), i.e., sputtering techniques, atomic layer deposition (ALD), or ion beam assisted deposition. These coating techniques allow the formation of coating surfaces with a surface roughness of less than about 20 micrometers to be uniformly coated.

In one embodiment, the thin film coating is deposited on the substrate using reactive magnetron sputtering of titanium in argon and an oxygen partial pressure, with substrate heating. Sputtering techniques in general are disclosed by Safi, “Recent aspects concerning DC reactive magnetron sputtering of thin films: a review,” Surface and Coatings Technology, 127:203-219 (2000), incorporated herein by reference. Techniques to deposit thin titanium oxide films specifically are disclosed by Guerin et al, “Reactive-sputtering of titanium oxide thin films,” Journal of Vacuum Science & Technology A, 15(3):712-715 (1997), incorporated herein by reference; Baroch et al, “Reactive magnetron sputtering of TiOx films,” Surface & Coatings Technology, 193:107-111 (2005), incorporated herein by reference; Barnes et al, “The mechanism of TiO2 deposition by direct current magnetron reactive sputtering,” Thin Solid Films, 446:29-36 (2004), incorporated herein by reference; and Barnes et al, “The mechanism of low temperature deposition of crystalline anatase by DC magnetron sputtering,” Surface & Coatings Technology, 190:321-330 (2005), incorporated herein by reference.

In a specific embodiment, to increase the coating adhesion, the substrate is first cleaned using conventional cleaning procedures before conducting the deposition process for forming the thin film coating. Further, the substrate may also be sputter etched before depositing the thin film coating, for example, in order to remove native oxide on a metal implant. Typically, such sputter etching is not employed in the manufacture of composite materials using ceramic and/or polymeric implants substrates.

If desired, the coating may be porous, and in one embodiment, may be nanoporous, having pores of a size in the range of 0.1-100 nm. Porosity of the coating may be controlled via the deposition process for example, by temperature and the partial pressures of argon and oxygen, mainly that of the argon pressure.

In a specific embodiment, a convenient method for depositing the thin film coating is by a sputtering technique, wherein the titanium dioxide phase of the deposited coating is controlled via the temperature of the substrate, typically using a temperature range of from greater than room temperature to below 400° C., in combination with the oxygen flux in the coating chamber. The partial pressure of oxygen in the coating chamber may, for example, be in the range of from about 0.01 to 5 Pa, preferably from about 0.1 to 1 Pa. The argon pressure range may be greater than 0.1 mTorr to 30 mTorr. The use of low temperature and high argon pressure results in a more porous film than does the use of a high temperature and low argon pressure. Accordingly, porosity can be controlled by adjusting the temperature and pressure parameters. In addition, the depth of the pores can be controlled by changing the reaction conditions at an appropriate time of the process. One of ordinary skill in the art will appreciate that the foregoing description is one of many deposition techniques known in the art that can be used in forming a thin film coating according to the present invention.

In a specific method of depositing the thin film coating to a surgical implant substrate, the coating adhesion may be increased by first depositing a Ti layer, or a layer containing Ti and N or any other suitable element M which in combination with Ti gives good adhesion on the substrate. In a more specific embodiment, pure Ti metal is first deposited on the surgical implant substrate. The partial pressure of oxygen may then be increased gradually to obtain a gradient coating going from the pure Ti metal, or TiM_{y} (y being equal to or less than 1) at the interface between the implant surface and the film coating to the desired crystalline titanium oxide phase at the thin film coating surface. In another embodiment, the method comprises co-sputtering an element M, for example carbon, and Ti under nitrogen and/or oxygen pressure to form a Ti(O₂₋ₓ,C_{y1},N_{y2}) film, where 0≦x<2, y₁+y₂=y, and 0≦y≦1. The partial pressure of nitrogen and oxygen and the sputter rate of carbon can be controlled to form any gradient structure in the film. In yet another embodiment, the thin film coating may be formed by evaporating Ti and sputtering C under nitrogen and/or oxygen pressure to form a Ti(O₂,C_{y1},N_{y2}) film, where x, y1 and y2 are as defined above. The partial pressure of nitrogen and oxygen and the sputter rate of carbon can be controlled to form any gradient structure in the film within the composition ranges.

In a specific embodiment of the invention, the thin film titanium oxide coating is deposited with porous properties and therefore is suitable for loading with a releasable agent having desired functional properties, such as, for example, an active pharmaceutical ingredient (drug), bio molecule, or ion, or the like, or a combination thereof. A surgical implant composite material including a releasable agent loaded in the thin film coating is advantageous for targeted and/or controlled release of the agent in vivo. The porous titanium oxide coating on a surgical implant substrate can be loaded with a releasable agent such as a drug, ion or bio molecule, or combinations thereof, via any loading technique known in the art. Examples of such techniques include, but are not limited to, soaking or vacuum impregnating the coating with a diluted suspension of the releasable agent for later delivery in vivo from the thin film coating. Other examples include absorption loading, solution loading, evaporation loading (e.g. rotary evaporation loading), solvent loading, air suspension coating techniques, precipitation techniques, spray-coagulation methods, or combinations thereof. Specific examples of releasable agents suitable for use in this embodiment include, but are not limited to, antibiotics, e.g., gentamicin, such as gentamicin sulfate, and other aminoglycosides such as amikacin, kanamycin, neomycin, netilmicin, paromomycin, streptomycin, tobramycin, and apramycin, bone morphogenesis proteins, peptides, bisphosphonates, opioids, opiates, vitamins anti-cancer drugs, iodine, Ag, combinations thereof, and the like. One of ordinary skill in the art will appreciate that the porosity of the thin film coating may be configured in order to provide a desired rate of release of the releasable agent therefrom.

In a further embodiment, the composite material comprising a surgical implant substrate and the thin film coating may further include a biomimetic coating provided on the surface of the thin film coating. Biomimetic coatings are known in the art and any suitable biomimetic may be employed herein. In one embodiment, the biomimetic coating comprises an apatite, for example, hydroxylapatite (HA), calcium phosphate, or the like. The thickness of the biomimetic coating may be selected based on the application of the composite material as desired. In one embodiment, wherein the composite material includes a surgical implant substrate, the biomimetic coating has thickness less than about 100 μm, and more specifically has a thickness less than about 50 μm. In a further embodiment, the biomimetic coating has a thickness less than about 5 μm. The composite material may be provided with the biomimetic coating by any suitable coating technique known in the art.

In one embodiment, the composite material comprising a surgical implant substrate and the thin film coating as described is immersed in a calcium phosphate buffer solution for biomimetic deposition of HA or Brushite (CaHPO₄.2H₂O) or a similar material. The deposition rate and structure is controlled via solution temperature, composition and solution strength. Brushite is deposited from buffer solutions with a pH of below 4.2 and HA or deficient HA is deposited from buffer solutions with a pH above 4.2. In a specific embodiment, HA is deposited via simulated body fluid or phosphate buffer saline. Preferably, the solution temperature is below 95° C., more specifically below 70° C., and more specifically at 37° C. For example, the titanium dioxide thin film coated implant substrate is immersed for a time period of up to several months in the solution, preferably up to 7 days, at 37° C.

Optionally, the biomimetic coating can be loaded with a releasable agent having desired functional properties as described above, via any loading technique known in the art, as discussed above. Such loading of the biomimetic coating is advantageous for targeted and/or controlled release of the agent in vivo. In one embodiment, the releasable agent can be loaded in parallel with the formation of the biomimetic coating.

Optionally, the loaded coating, either the thin film coating or the biomimetic coating, may in turn be coated with a resorbable polymer for extended release of the release agent. The resorbable polymer can be applied via any suitable technique, for example by solution, melting or spraying onto the coated substrate, and drying or solidifying. The thickness of the polymer layer is suitable below 200 μm, preferably below 50 μm. Examples of such polymers include, but not limited to, polylactic acids, propylene fumarate and chitosan. Optionally, cyclodextrin can be used to obtain slow release of the releasable agent as well.

In another aspect of the invention, a kit is provided comprising a surgical implant composite material comprising a surgical implant substrate and a thin film coating of titanium oxide as described herein, optionally including a biomimetic coating, together with one or more solutions of a releasable agent having a desired functional property, i.e., an active pharmaceutical ingredient, an ion or a biomolecule. The solution is operable to load the releasable agent onto the surgical implant composite material upon contact of the solution with the surgical implant composite material. Thus, prior to implantation, a specific composite material may be combined with a selected releasable agent based on the needs of the specific patient for which the implant is intended.

Various embodiments of the composite materials and methods of the invention are described in the following examples.

### Example 1

This example demonstrates a composite material according to the invention. To provide a self-cleaning glass, for example, a window, a very thin layer of TiO₂ is integrated onto the glass surface while the glass is still in the molten state, so that the TiO₂ will not wear from the window. In one embodiment of the present invention, the window glass is coated by depositing a thin film coating having a Ti oxide gradient structure in which the oxygen content of the coating is lower close to the glass substrate than at the external thin film coating surface in order to promote adhesion and wear resistance. For self-cleaning windows and other types of photocatalytic applications, it is preferred that the outermost part of the TiO₂ containing surface is crystalline.

### Example 2

In photovoltaic applications such as in both wet and solid Grätzel solar cells, one of the active parts consists of a TiO₂ containing layer attached to a charge collecting electrode substrate. Usually this layer is formed in a sol-gel process. In one embodiment of the present invention, the electrode substrate is coated by depositing a thin film coating having a Ti oxide gradient structure in which the oxygen content of the coating is lower closer to the electrode substrate than at the thin film coating surface in order to promote adhesion, wear resistance and/or electric transport. For photoelectrochemical solar energy conversion, the TiO₂ thin film is advantageously nanocrystalline or polycrystalline.

### Example 3

In electrochromic devices, TiO₂ may be used as an active electrochromic layer that changes colour upon ion (and concomitant electron) intercalation. In such applications, the TiO₂ is normally deposited onto a transparent electronically-conducting substrate. In one embodiment of the present invention, the electronic conductor in an electrochromic device is coated by depositing a thin film coating having a Ti oxide gradient structure in which the oxygen content of the coating is lower closer to the substrate than at the thin film coating surface facing the ion-conducting electrolyte in order to promote adhesion. In this type of application, it is important to keep the low oxygen content part of the coating thin enough to avoid loss of optical transparency. For electrochromic applications, the Ti oxide thin film coating is advantageously amorphous or anatase.

### Example 4

In one example, a surgical implant substrate having a sputter deposited thin film coating of nanocrystalline rutile TiO₂, without a gradient structure, is immersed in a simulated body fluid for seven days at a temperature of 60° C. to form a biomimetic coating of hydroxylapatite (HA) thereon. An active substance, for example gentamicin, is then soaked into the HA biomimetic layer formed on the surface. In another example, HA is allowed to form nuclei on the TiO₂ thin film coating during only 48 hours of storing the substrate in simulated body fluid at 37° C. After that, an active substance, for example gentamicin, is mixed with the simulated body fluid and allowed to co-precipitate and/or co-crystallize with the HA. In yet another example, a thin film of HA is allowed to form on the TiO₂ thin film coating, after which the composite material is alternately soaked in two separate solutions, one solution containing the active substance and the other solution containing simulated body fluid until the desired thickness of the HA-containing biomimetic layer was formed.

### Example 5

A series of experiments were performed to deposit titanium oxide coatings on substrates. Specifically, graded titanium dioxide thin films were prepared in a reactive DC magnetron sputtering unit (Balzer UTT400). The coatings in these experiments were deposited on grade 2 Ti implants using the sputtering conditions as described below. The working chamber was mounted with a turbo molecular pump (TMU 521P). The chamber had a base pressure of approximately 10⁻⁷ mbar after backing. Argon and oxygen were sprayed into the chamber from different nozzles controlled by a mass flow controller (Bronkhorst Hi-Tec). The pressure in the chamber was adjusted by a manual valve mounted between the chamber and the pump and was measured by a capacitance diaphragm gauge (model CMH-01S07). The sample holder was rotated. A pure titanium target (99.9%, 2″ diameter*0.25″ thick bought from Plasmaterials) was used for depositing a thin film layer. Pure argon (99.997%) and oxygen (99.997%) were used for the reactive sputtering. Sputtering conditions were as follows: current of 900 mA, effect of 330 W, pressure of 20 mTorr, oxygen gas flow of 0-4 ml/min (to form the gradient structure), and argon gas flow of 100 ml/min.

A first experiment (Experiment 1) was conducted by first depositing a layer of pure titanium of 10 nm thickness. On the surface of this pure titanium layer a second layer of 50 nm was formed with the oxygen flow gradually increasing from near zero to a constant value to give an oxygen content gradient in the resulting Ti oxide layer. When the oxygen flow was high enough to produce TiO₂, the flow was held constant at this flow to form a 200 nm thick TiO₂ layer. The substrate temperature during these steps was held constant at 350° C. The resulting material is referred to as Sample 1.

In a second experiment (Experiment 2), similar coatings as described above were deposited without substrate heating to form Sample 2.

A third experiment (Experiment 3), forming only the Ti layer and the TiO₂ layer thereon, without the gradient structure in between, was also performed. The substrate temperature during these steps was held constant at 350° C. to form Sample 3.

The obtained coatings were characterized using X-ray diffraction for phase composition (detection of crystalline phases), scanning electron microscopy for studying the film thickness in cross-section (LEO 440), and XPS for detecting the gradient structure. The coating adhesion was measured using Rockwell C indentation.

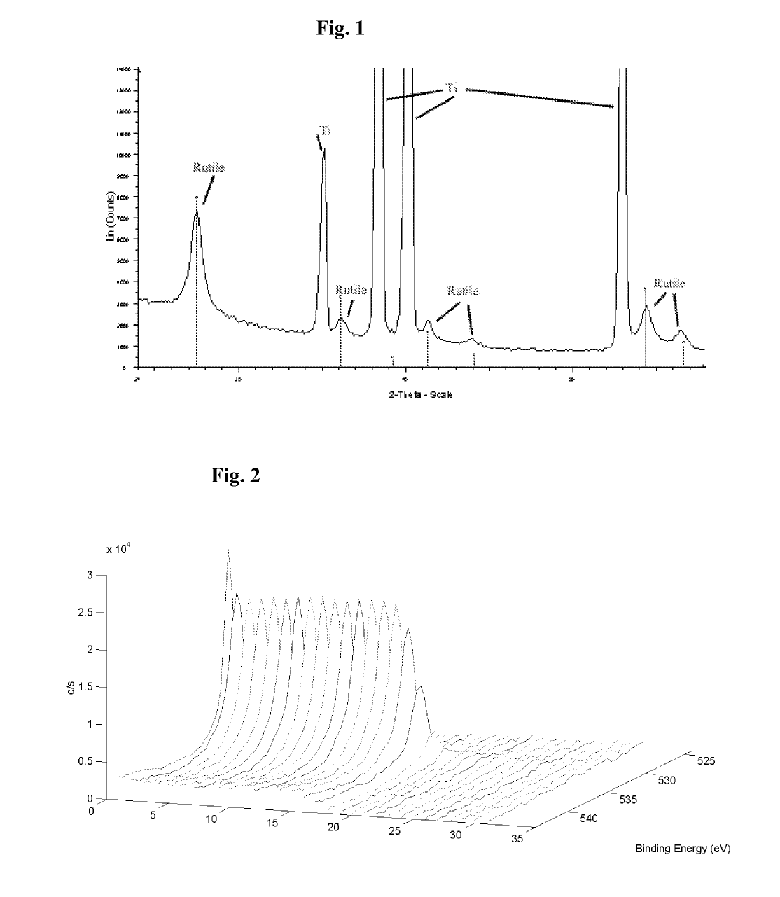

The coatings in Samples 1 and 2 resulting from experiments 1 and 2 were about 260 nm thick, while the coating in Sample 3 resulting from Experiment 3 was about 210 nm. The outermost region of the coatings deposited at 350° C., Samples 1 and 3, were nanocrystalline. As shown in FIG. 1, Sample 1 contained rutile phase TiO₂. The spectrum for the non-graded coating of Sample 3 was similar. The coating of Sample 2, deposited without heating, was amorphous and only peaks from the Ti substrate could be detected.

XPS analysis of the gradient coatings shows that the gradient zones in the coatings produced in Samples 1 and 2 of Experiments 1 and 2 were about 50 nm as is evident from the XPS of Sample 1 shown in FIG. 2.

Substrate adhesion testing using Rockwell C indentation showed that the adhesion was the highest for the gradient coating deposited using substrate heating, Sample 1.

These experiments show that the titanium oxide-containing coating having a gradient in the oxygen content and deposited at 350° C. (Sample 1) was nanocrystalline and had a higher adhesion as compared with titanium oxide-containing coating with no gradient (Sample 3) and as compared with gradient coating deposited at ambient temperature (Sample 2).

### Example 6

Samples 1-3 from Example 5 were tested for their in vitro bioactivity following the method of Kokubo et al, “How useful is SBF in predicting in vivo bone bioactivity?,” Biomaterials, 27:2907-2915 (2006).

Soaking bone bioactive materials in simulated body fluid (SBF) or phosphate buffered saline (PBS) results in the formation of a biomimetic HA coating on the surface. Thus, the titanium oxide coated implants from Example 5 were ultrasonically cleaned, at a resonance of 20 kHz, by the following procedure: 5 min each in a bath of acetone, ethanol and finally deionized water. The coated Ti implants were then immediately immersed in 40 ml of 37° C. preheated phosphate buffered saline (PBS, Dulbecco's Phosphate Buffered Saline, Sigma-Aldrich Company Ltd.) in falcon tubes. The presence of HA was detected using thin film X-ray diffraction and scanning electron microscopy (SEM).

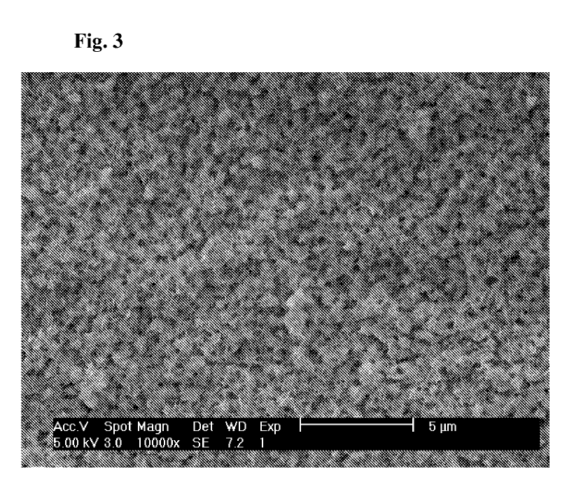

After 7 days in the PBS solution a uniform, porous biomimetic hydroxylapatite layer was formed on the nanocrystalline titanium dioxide surface of Sample 1, as shown in FIG. 3, and of Sample 3. The coating of Sample 2 deposited at ambient temperature did not show a biomimetic hydroxylapatite layer on the surface.

The crystalline coatings were therefore bioactive and a biomimetic HA coating was formed. The amorphous coating was not bioactive.

### Example 7

A series of drug loading experiments were performed on the HA coated crystalline titanium oxide coatings from Example 6. Specifically, the drug loading employed the antibiotic drug gentamicin sulfate (GS), incorporated in the hydroxylapatite layer (HA). All tests were performed at 37° C. in falcon tubes. Two soaking procedures were tested:
Soaking the HA coatings from Example 6 in a water-GS solution for 3 days (Experiment 4).
Soaking the HA coatings from Example 6 in a PBS-GS solution for 7 days (Experiments 5). This allows a co-precipitation and/or co-crystallization of both GS and HA.

In a second series of experiments ethanol was added to the solution (10 wt %) to reduce the solubility of GS. These experiments were denoted 6 and 7.

The release of GS from the implants was studied using Staphylococcus aureus, Staphylococcus epidermidis, and Pseudomonas aeruginosa bacteria. All strains were grown overnight on Muller-Hinton (Difco) agar plates at 37° C. Samples 4-7, resulting from Experiments 4-7, respectively, were put into the agar plates and the diameter of the bacteria death surrounding the samples was measured as a function of time.

All samples showed antibacterial effect for more than 24 hours with Samples 6 and 7 showing a wider effected diameter than Samples 4 and 5. Samples 6 and 7 also had a longer release period than Samples 4 and 5.

These experiments showed a slow drug release from the HA/crystalline coatings described in Examples 4 and 5.

### Example 8

In one example, the surface of a surgical implant substrate was sputter coated with a thin layer of pure Ti (20 nm). On the surface of this layer, a Ti oxide layer with oxygen content increasing of 0 to 2 was sputter deposited. The thickness of the resulting gradient layer was 70 nm. As an outermost bioactive coating, a layer of nanocrystalline TiO₂ was sputtered (100 nm). The coated implant surface was soaked in PBS to form a biomimetic HA layer. The resulting material was packed, sealed and sterilized using gamma radiation. The sterilized implant was soaked with a liquid solution containing amoxicillin for 15 minutes. The effect of the drug was studied in vitro using the standard agar plate with bacteria. The soaked implant showed an antibacterial effect for more than 24 hours. The results showed that prolonged antibacterial effect of the implant can be achieved by soaking an implant including a porous biomimetic HA layer immediately prior to implantation.

The specific illustrations and embodiments described herein are exemplary only in nature and are not intended to be limiting of the invention defined by the claims. Further embodiments and examples will be apparent to one of ordinary skill in the art in view of this specification and are within the scope of the claimed invention.

1. A surgical implant composite material comprising a surgical implant substrate and a thin film coating deposited on the substrate, the thin film coating comprising TiO₂₋ₓM_{y}, wherein M is one or more elements which does not adversely effect adherence of the coating to the substrate, y is the sum of the mols of all M elements, 0≦x<2 and 0≦y≦1, and wherein an outermost portion of the thin film coating is crystalline.

2. A surgical implant composite material according to claim 1, wherein the thin film coating has a gradient composition through at least a portion of the thin film coating thickness and wherein the thin film coating is substantially free of oxygen at the substrate surface.

3. A surgical implant composite material according to claim 2, wherein the portion of the thin film coating thickness having the gradient composition is greater than about 7 nm.

4-36. (canceled)

37. A surgical implant composite material according to claim 2, wherein the gradient composition increasing in oxygen content in a direction extending from the substrate toward the thin film coating surface.

38. A surgical implant composite material according to claim 1, wherein the thin film coating has a thickness less than about 30 μm.

39. A surgical implant composite material according to claim 1, wherein M is selected from the group consisting of C, N, Ag, I, Si, Ca, Zr, Hf and P.

40. A surgical implant composite material according to claim 1, wherein the substrate is metallic.

41. A surgical implant composite material according to claim 1, wherein the thin film coating is loaded with a releasable agent comprising an active pharmaceutical ingredient, ion or bio molecule, or a combination thereof.

42. A surgical implant composite material according to claim 1, further comprising a biomimetic coating comprising apatite or calcium phosphate, with a thickness less than about 100 μm, on the thin film coating.

43. A surgical implant composite material according to claim 42, wherein the biomimetic coating comprises hydroxylapatite.

44. A surgical implant composite material according to claim 42, wherein the biomimetic coating is loaded with a releasable agent comprising an active pharmaceutical ingredient, ion or bio molecule, or a combination thereof.

45. A kit comprising a surgical implant composite material according to claim 1 and at least one solution of a releasable agent comprising an active pharmaceutical ingredient, ion or bio molecule, or a combination thereof, the solution being operable to load the releasable agent onto the surgical implant composite material upon contact of the solution with the surgical implant composite material.

46. A kit comprising a surgical implant composite material according to claim 42 and at least one solution of a releasable agent comprising an active pharmaceutical ingredient, ion or bio molecule, or a combination thereof, the solution being operable to load the releasable agent onto the surgical implant composite material upon contact of the solution with the surgical implant composite material.

47. A method of forming a surgical implant composite material, comprising depositing by physical vapor deposition technique or chemical vapor deposition technique a thin film coating on a surgical implant substrate, the thin film coating comprising TiO₂₋ₓM_{y}, wherein M is one or more elements which does not adversely effect adherence of the coating to the substrate, y is the sum of the mols of all M elements, 0≦x<2 and 0≦y≦1, and wherein an outermost portion of the thin film coating is crystalline.

48. A method according to claim 47, further comprising loading the thin film coating with a releasable agent comprising an active pharmaceutical ingredient, ion or bio molecule, or a combination thereof.

49. A method according to claim 47, further comprising forming a biomimetic coating on the thin film coating.

50. A method according to claim 49, further comprising loading the biomimetic coating with a releasable agent comprising an active pharmaceutical ingredient, ion or bio molecule, or a combination thereof.
